(19) United States
(12) Patent Application Publication
(10) Pub. No.: US 2023/0190255 A1
(43) Pub. Date: Jun. 22, 2023

(54) PATIENT POSITIONER

(71) Applicant: GLOBUS MEDICAL, INC., Audubon, PA (US)

(72) Inventor: Phillip Levins, Conshohocken, PA (US)

(21) Appl. No.: 17/559,325

(22) Filed: Dec. 22, 2021

(51) Int. Cl. A61B 17/02

(52) U.S. Cl. CPC A61B 17/025; A61B 2017/0256

(57) ABSTRACT

Systems for positioning a patient are provided. A patient positioning system comprises a set of break assemblies, each break assembly comprising a mount for receiving padding, wherein each break assembly is operable to independently rotate to provide a coronal break relative to a longitudinal axis of the frame; and a set of tilt assemblies mounted to the frame, each tilt assembly operable to independently tilt relative to a longitudinal axis of the frame, wherein the break assemblies are mounted to the tilt assemblies.

### BACKGROUND

Prone lateral lumbar interbody fusion (LLIF) may be performed on an open Jackson frame. However, traditional Jackson frames do not allow certain patient manipulation movements needed to improve access and ergonomics of LLIF in the prone position. Anterior-to-Psoas (ATP) access is also restricted using a traditional Jackson frame. In traditional lateral decubitus LLIF, surgeons often “break the bed” to put an angle between the ribs and iliac crest. This opens the space between the hips and ribs to improve access to difficult levels such as L4-5 and L1-2, especially in patients with challenging anatomy. With the patient positioned prone on a Jackson frame, however, there is currently no way to controllably induce coronal break on the patient. Lateral surgery in the prone position often has poor ergonomics. Jackson frame height and tilt limitations, as well as surgeon height/stature may lead to reduced visualization of the surgical corridor and uncomfortable working angles. There is a need to improve surgeon ergonomics by increasing control over patient height and tilt. LLIF requires true anteroposterior (AP) and lateral C-arm imaging.

Standard Jackson frames only allow about 25 degrees of tilt. Standard C-arm devices are unable to achieve a true lateral shot with the patient on a tilted Jackson frame table, until about 35 degrees of table tilt. The reasoning for this is that to see a direct lateral image the C-arm would need to “rainbow” over the patient about 65 degrees or more, which many devices are incapable of doing. However, increasing total patient tilt to about 40 to 45 degrees allows a C-arm to get a true lateral image by rainbowing over the patient. Therefore, with existing equipment, the surgeon would need to de-tilt the patient for each lateral fluoro shot. An additional challenge to prone position lateral access is that it currently does not allow for an oblique or Anterior-to-Psoas (ATP) approach to the spine. The Jackson frame rail directly blocks the oblique trajectory that would be needed for proper ATP technique. Tilting the Jackson frame does not solve this issue because the frame rail remains in the same position relative to the patient. There is a need to increase patient tilt relative to the Jackson frame rail to facilitate ATP access.

### SUMMARY

In an exemplary embodiment, the present disclosure provides a patient positioning system comprising a set of break assemblies, each break assembly comprising amount for receiving padding, wherein each break assembly is operable to independently rotate to provide a coronal break relative to a longitudinal axis of the frame; and a set of tilt assemblies mounted to the frame, each tilt assembly operable to independently tilt relative to a longitudinal axis of the frame, wherein the break assemblies are mounted to the tilt assemblies.

In another exemplary embodiment, the present disclosure provides a patient positioning system comprising: a frame; and a set of break assemblies, each break assembly comprising amount for receiving padding, wherein each break assembly is operable to independently rotate to provide a coronal break relative to a longitudinal axis of the frame.

In another exemplary embodiment, the present disclosure provides a patient positioning system comprising: a frame; and a set of tilt assemblies mounted to the frame, each tilt assembly operable to independently tilt to adjust a padding mount relative to a longitudinal axis of the frame.

It is to be understood that both the foregoing general description and the following detailed description are exemplary and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTION OF THE DRAWINGS

These drawings illustrate certain aspects of some of the embodiments of the present disclosure and should not be used to limit or define the disclosure.

FIG. 1A illustrates a breaking assembly for positioning a patient, in a neutral position, in accordance with particular embodiments of the present disclosure;

FIG. 1B illustrates the breaking assembly with a coronal break, in accordance with particular embodiments of the present disclosure;

FIG. 2A-2C illustrate close-up views of the breaking assembly, in accordance with examples of the present disclosure;

FIGS. 3A-3D illustrate top views of the breaking assembly, in accordance with examples of the present disclosure;

FIGS. 4A-4E illustrate a tilt assembly, in accordance with examples of the present disclosure;

FIGS. 5A-5C illustrates tilt assemblies mounted to a Jackson frame;

FIGS. 6A and 6B illustrate the break assembly mounted to the tilt assembly, in accordance with examples of the present disclosure; and

FIGS. 7A-7C illustrate the break assemblies and tilt assemblies mounted to the Jackson frame, in accordance with examples of the present disclosure.

### DETAILED DESCRIPTION

For the purposes of promoting an understanding of the principles of the present disclosure, reference will now be made to the implementations illustrated in the drawings and specific language will be used to describe them. It will nevertheless be understood that no limitation of the scope of the disclosure may be intended. Any alterations and further modifications to the described devices, instruments, methods, and any further application of the principles of the present disclosure are fully contemplated as would normally occur to one skilled in the art to which the disclosure relates. In particular, it may be fully contemplated that the features, components, and/or steps described with reference to one or more implementations may be combined with the features, components, and/or steps described with reference to other implementations of the present disclosure. For simplicity, in some instances the same reference numbers are used throughout the drawings to refer to the same or like parts.

Embodiments generally relate to spinal surgery. More particularly, embodiments relate to systems for inducing a coronal break (e.g., lateral directions) and tilt (e.g., vertical directions) through worm gear connections to improve lateral access in the prone position; improve surgeon ergonomics for lateral access in the prone position; allow ATP access in the prone position; reduce scoliosis deformity from positioning; and reduce axial rotation deformity from positioning. The worm gear connection may include any suitable gear arrangement such as a worm drive in which a worm (a gear in the form of a screw) meshes with a worm wheel (e.g., a spur gear). The two elements may also be referred to as the worm screw and worm gear.

Each break assembly may include a first pad/padding mount (e.g., to receive padding configured to receive the chest region of a patient in a prone position) or a second pad mount (e.g., to receive padding configured to receive the hip/thigh region of the patient in the prone position). The mounts may include side walls to receive the padding.

The first pad mount and the second pad mount may rotate independently relative to a longitudinal axis. This independent rotation allows for the coronal break to occur. In the neutral position, the mounts may be aligned with one another along the axis (e.g., both pads at 0 degrees of rotation). The structure of the mounts may be similar or different from each other. The mounts may include any suitable shape or size to accommodate the padding/cushion to receive a patient in a prone position. For example, each mount may also include side walls for mounting to the frame including rails.

During the break, each of the mounts may be rotated relative to the axis (e.g., lateral rotation) causing the break in the alignment. For example, the mounts may be angled relative to the axis during the break. In addition to the mounts, each break assembly may further include a base, a bearing, a worm gear, a worm, and a worm shaft, all of which may be of any suitable shape or size. The base may include side walls for mounting to the frames. In some examples, the base mounts to the Jackson frame. The bearing connects the base to the worm gear allowing the worm gear to rotate with respect to the base. The mount is rigidly connected to the worm gear. This allows the mount to swivel/rotate relative to the base/Jackson frame rails. The worm is mounted on the shaft (e.g., a drive shaft), controlled by a handle at the side of the bed or a powered motor.

As a user turns the shaft, the worm drives the worm gear which in turn causes the mount (and associated pad) to swivel about the axis relative to the base. This mechanism may be applied to both the hip and chest padding mounts to allow for an increased amount of break. fit some examples, the worm gear and the bearing may be mounted with buffer plates to allow sufficient clearance for the worm. The independent rotation allows for the coronal break to occur. In the neutral position, the mounts may be aligned with one another along the axis (e.g., both pads at 0 degrees of rotation). The worm gear connection allows rotation. Also, increasing axial tilt may improve surgeon ergonomics and allow Anterior-to-Psoas (ATP) lateral access to the spine. The tilt assembly drives axial tilt (e.g., vertical directions) relative to the longitudinal axis of the Jackson frame, with a worm gear connection.

For example, the tilt assembly is comprised of a housing, a geared swing plate, a worm, and support rollers all of which may be of any suitable size or shape. The worm may be driven either with a manual handle or a powered motor. This interfaces with the toothed/geared swing plate, driving the tilting motion.

Rollers support the load while allowing motion. The housing includes a track that mates with a guide feature (e.g., protrusion/ridge) on the swing plate which prevents the swing plate from lifting up from the housing. To facilitate the swinging motion, the geared swing plate includes rails on the outer side adjacent to the housing, and a worm gear mounted along the center plane. A worm is mounted in the center plane of the housing to drive the geared swing plate. In other embodiments, the work gear may be offset from the center plane of the housing.

The assemblies are to be mounted to a Jackson frame. Tilting both assemblies in the same direction improves surgeon ergonomics and allows ATP access to the spine (by increasing height of the patient relative to the Jackson frame rails). Asymmetrically tilting each assembly may allow surgeons to correct axial rotational deformities by rotating the patient spine in the direction opposite the deformity. To facilitate axial deformity reduction, the assembly 400 rotates around the long axis of the patient spine.

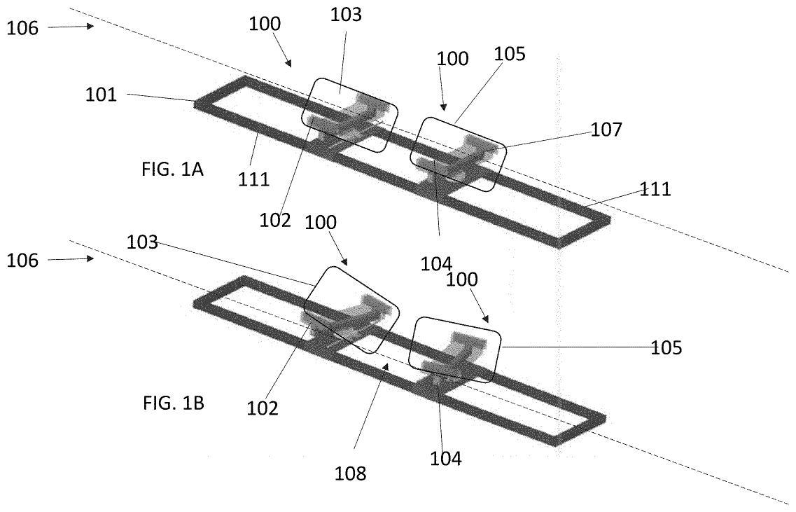

FIG. 1A illustrates coronal break assemblies 100 in neutral positions, in accordance with particular embodiments of the present disclosure. In the neutral positions, there is no rotation (e.g., zero degrees of rotation) of each of the assemblies 100. Standard C-arm devices are unable to achieve a true lateral shot with the patient on a tilted Jackson frame table, until about 35 degrees of table tilt. Lateral surgery in the prone position often has poor ergonomics. Jackson frame height and tilt limitations, as well as surgeon height/stature may lead to reduced visualization of the surgical corridor and uncomfortable working angles. There is a need to improve surgeon ergonomics by increasing control over patient height and tilt. LLIF requires true anteroposterior (AP) and lateral C-arm imaging.

In some examples, each break assembly 100 may be mounted to a frame 101 (e.g., a Jackson frame or any other suitable frame for surgery). The frame 101 may extend lengthwise to accommodate/support a patient during surgery. In some examples, the frame may include rails and may be made out of a rigid material such as for example, metal.

Each break assembly 100 may include a first pad/padding mount 102 (e.g., to receive padding 103 configured to receive the chest region of a patient in a prone position) or a second pad mount 104 (e.g., to receive padding 105 configured to receive the hip/thigh region of the patient in the prone position). The mounts 102 and 104 may include side walls 107 to receive the padding 103 and 105. Each mount may be made of any suitable material such as metal for example. The mounts may be shaped for example with multiple sides to secure padding to the mounts with fasteners/welds.

The first pad mount 102 and the second pad mount 104 may rotate independently relative to a longitudinal axis 106. This independent rotation allows for the coronal break to occur. In the neutral position, the mounts 102 and 104 may be aligned with one another along the axis 106 (e.g., both pads at 0 degrees of rotation). The structure of the mounts may be similar or different from each other. The mounts 102 and 104 may include any suitable shape or size to accommodate the padding/cushion to receive a patient in a prone position. For example, each mount 102/104 may also include side walls 109 for mounting to the frame 101 including rails 111.

FIG. 1B illustrates the break assemblies 100 with a coronal break 108, in accordance with particular embodiments of the present disclosure. During the break 108, each of the mounts 102 and 104 may be rotated relative to the axis 106 (e.g., lateral rotation) causing the break 108 in the alignment. For example, the mounts may be angled relative to the axis 106 during the break. During the break, each of the mounts may be rotated relative to the axis (e.g., lateral rotation) causing the break in the alignment. For example, the mounts 102 and 104 may be angled relative to the axis during the break. The mounts 102 and 104 swivel/rotate relative to the base/Jackson frame rails 111. This allows bending of the patient during surgery.

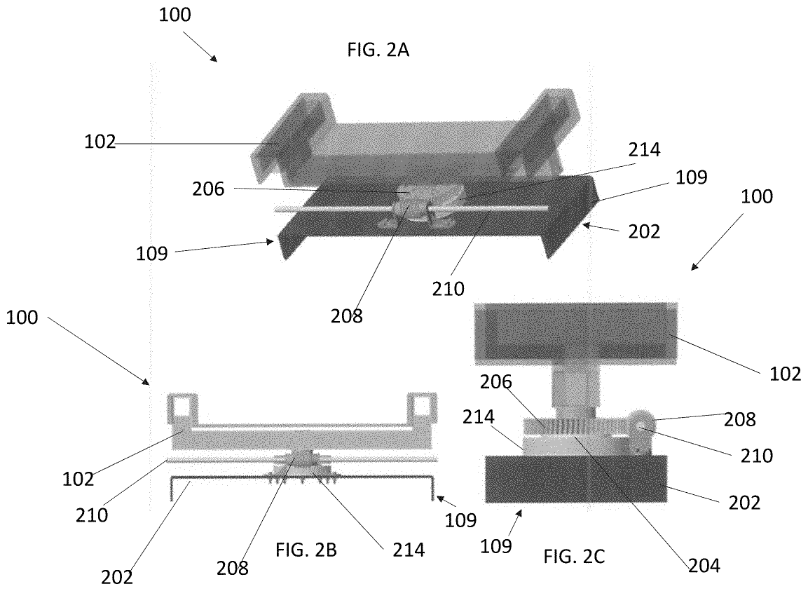

FIG. 2A-2C illustrate close-up views of the break assemblies 100 including the mount 102, in accordance with examples of the present disclosure. The coronal break reduces scoliosis deformity from positioning. Also, the coronal break improves lateral access when the patient is in the prone position. It should be noted that additional mounts may be configured similarly.

The break assembly 100 may be operable to rotate (e.g., laterally) the mount 102. In addition to the mount 102 (or mount 104), each break assembly 100 may further include a base 202, a bearing 204, a worm gear 206, a worm 208, and a worm shaft 210, all of which may be of any suitable shape or size. The base 202 may include side walls 109 for mounting to the frame (shown on FIG. 1). The worm gear connection may include any suitable gear arrangement such as a worm drive in which a worm (a gear in the form of a screw) meshes with a worm wheel (e.g., a spur gear). The two elements may also be referred to as the worm screw and worm gear.

In some examples, the base 202 mounts to the Jackson frame. As best shown on FIG. 2C, the bearing 204 connects the base 202 to the worm gear 206 allowing the worm gear 206 to rotate with respect to the base 202. The mount 102 is rigidly connected to the worm gear 206. This allows the mount 102 to swivel/rotate relative to the base 202/Jackson frame rails (shown on FIGS. 1A and 1B). The worm 208 is mounted on the shaft 210 (e.g., a drive shaft), controlled by a handle at the side of the bed or a powered motor.

As a user turns the shaft 210, the worm 208 drives the worm gear 206 which in turn causes the mount 102 (and associated pad) to swivel about the axis 106 (see FIG. 1) relative to the base 202. This mechanism may be applied to both the hip and chest padding mounts 102 and 104 to allow for an increased amount of break. In some examples, the worm gear 206 and the bearing 204 may be mounted with buffer plates 214 to allow sufficient clearance for the worm 208.

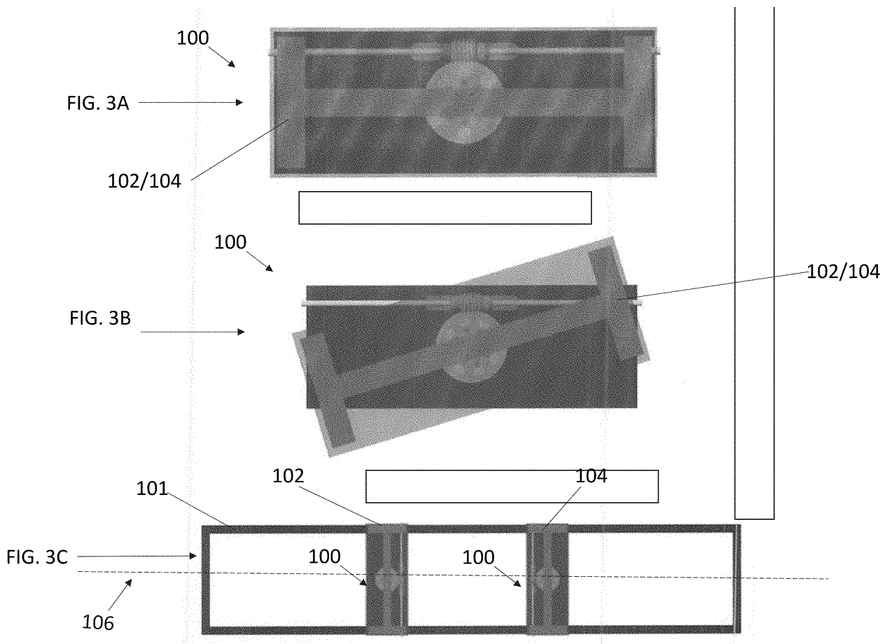

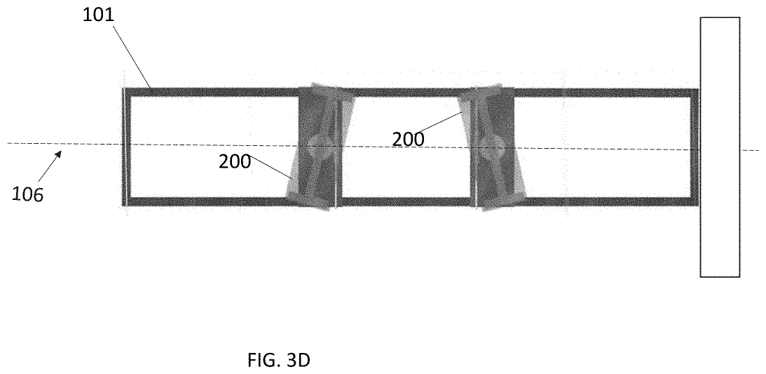

FIGS. 3A-3D illustrate top views of the break assembly 100, in accordance with examples of the present disclosure. FIG. 3A illustrates the neutral position (no angle) of the assembly 100. For example, the break assembly 100 has not been broken and is aligned along the longitudinal axis of the frame (e.g., axis 106 and frame 101 shown on FIG. 1A). The mount 102/104 has not been rotated leaving the patient in a neutral position.

FIG. 3B illustrates the broken position (angled) of the assembly 100. For example, the break assembly 100 has been broken and is no longer aligned along the longitudinal axis of the frame (e.g., frame 101 on FIG. 1A). The mount 102/104 has been rotated leaving the patient in a broken position. FIG. 3C illustrates the Jackson frame 101 with the assemblies 100 in the neutral position (unbroken). For example, both break assemblies 100 have not been broken and are aligned with each other along the longitudinal axis 106 of the frame 101. The mounts 102/104 have not been rotated leaving the patient in a neutral/unbroken position.

FIG. 3D illustrates the Jackson frame 101 with the assemblies 100 in the broken position. For example, both break assemblies 100 have been broken and are no longer aligned with each other along the longitudinal axis 106 of the frame 101. The rotation angles for the assemblies 100 may be similar or different. The mounts 102/104 have been rotated leaving the patient in a broken position.

The first pad mount 102 and the second pad mount 104 may rotate independently relative to a longitudinal axis 106. This independent rotation allows for the coronal break to occur. In the neutral position, the mounts 102 and 104 may be aligned with one another along the axis 106 (e.g., both pads 102 and 104 at 0 degrees of rotation). The worm gear connection, as illustrated in FIGS. 2A-2C allows rotation of the assemblies 100.

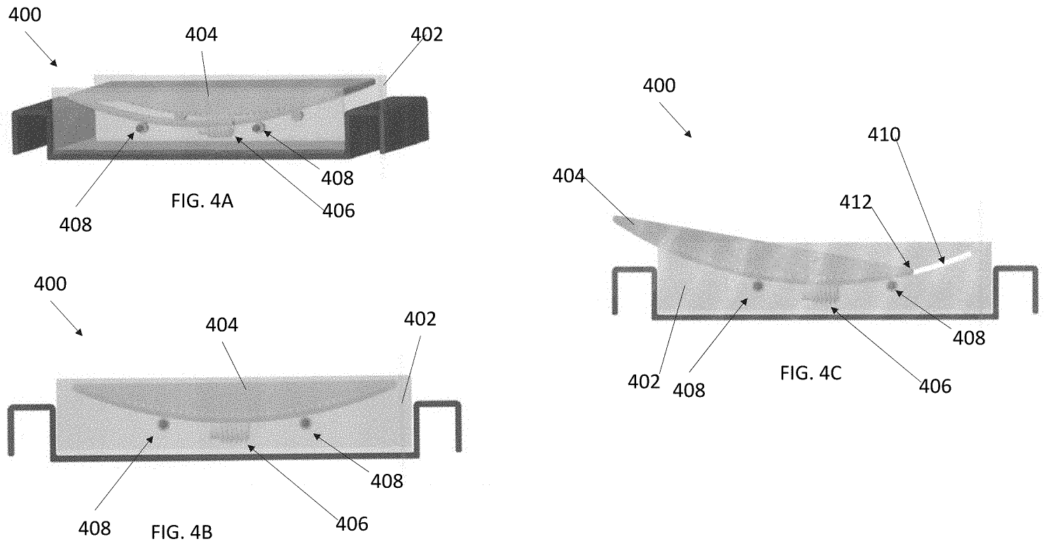

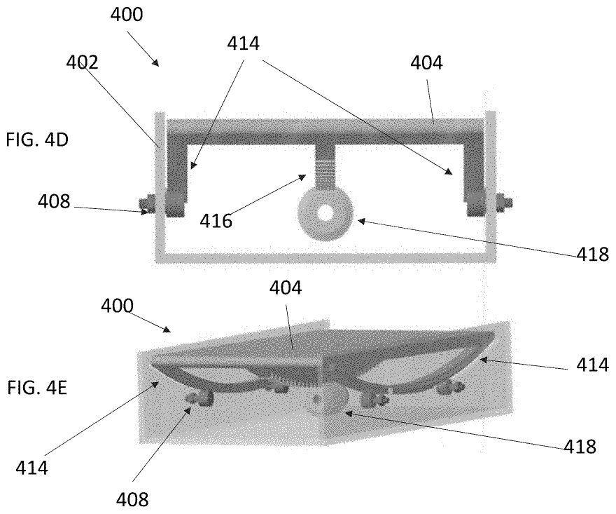

FIGS. 4A-4E illustrate a tilt assembly 400, in accordance with examples of the present disclosure. Typically, standard Jackson frames are typically limited to 25 degrees of patient tilt. Increasing axial tilt may improve surgeon ergonomics and allow Anterior-to-Psoas (ATP) lateral access to the spine. Tilting the Jackson frame does not solve this issue because the frame rail remains in the same position relative to the patient. There is a need to increase patient tilt relative to the Jackson frame rail to facilitate ATP access.

The tilt assembly 400 drives axial tilt (e.g., vertical directions) relative to the longitudinal axis of the Jackson frame, with a worm gear connection. For example, the tilt assembly 400 is comprised of a housing 402, a geared swing plate 404, a worm 406, and support rollers 408 all of which may be of any suitable size or shape. The worm 406 may be driven either with a manual handle or a powered motor. This interfaces with the toothed/geared swing plate 404, driving the tilting motion. The assembly 400 allows axial tilt to improve lateral access when the patient is in the prone position. The axial tilt allows ATP access in the prone position.

Rollers 408 support the load while allowing motion. Any suitable rollers may be employed for example, cylinders. The worm gear connection may include any suitable gear arrangement such as a worm drive in which a worm (a gear in the form of a screw) meshes with a worm wheel (e.g., a spur gear). The two elements may also be referred to as the worm screw and worm gear. As best shown on FIG. 4C, the housing 402 includes a track 410 that mates with a guide feature 412 (e.g., protrusion/ridge) on the swing plate 404 which prevents the swing plate 404 from lifting up from the housing 402. The track 4 (0 may be any suitable shape to prevent the swing plate 404 from lifting up from the housing 402. In some examples, the track 410 may include a U-shape.

As best shown on FIG. 4D, to facilitate the swinging motion, the geared swing plate 404 includes rails 414 on the outer side adjacent to the housing 402, and a worm gear 416 mounted along the center plane. A worm 418 is mounted in the center plane of the housing 402 to drive the geared swing plate 404. The assemblies 400 are to be mounted to a Jackson frame.

Tilting both assemblies 400 in the same direction improves surgeon ergonomics and allows ATP access to the spine (by increasing height of the patient relative to the Jackson frame rails). Asymmetrically tilting each assembly 400 may allow surgeons to correct axial rotational deformities by rotating the patient spine in the direction opposite the deformity. To facilitate axial deformity reduction, the assembly 400 rotates around the long axis of the patient spine.

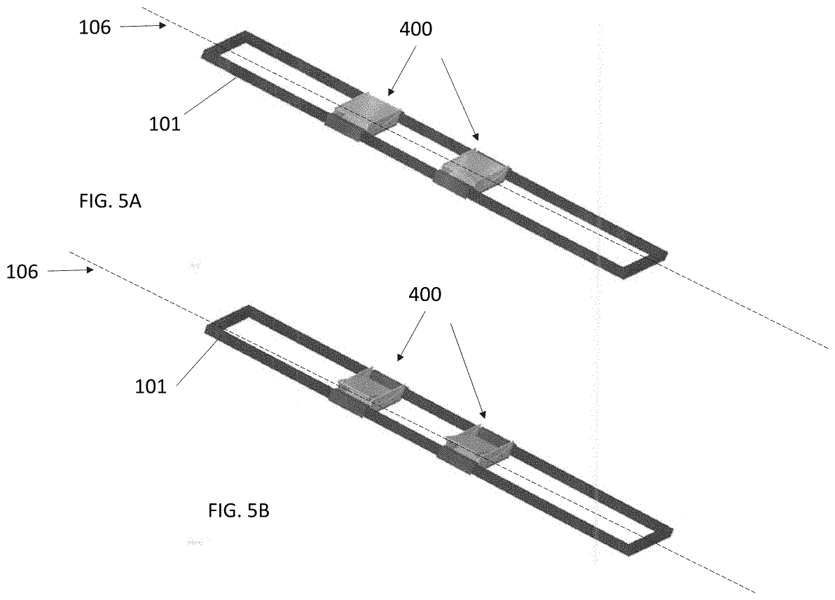

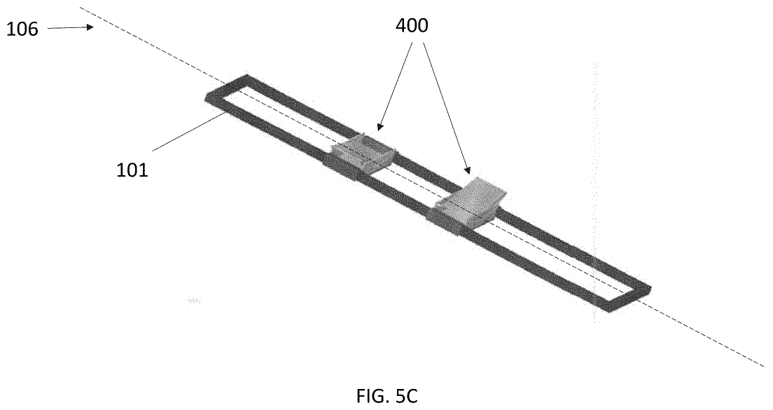

FIGS. 5A-5C illustrates the assemblies 400 mounted to the Jackson frame 101, in accordance with examples of the present disclosure. FIG. 5A illustrates the assemblies 400 in neutral positions (e.g., no tilt). For example, each assembly 400 may not include any tilt relative to the axis 106. In some examples, the tilt angles may be zero in the neutral position. FIG. 5B illustrates the assemblies 400 in symmetric positions. For example, both assemblies 400 may include the same tilt relative to the axis 106. FIG. 5C illustrates the assemblies 400 in asymmetric positions. For example, each assembly 400 may be tilted opposite to the other assembly, relative to the axis 106. The opposite tilt angles may be the same angle relative to the axis 106. For example, one assembly 400 may be tilted at −10 degrees and the other assembly 400 may be tilted at 10 degrees. In some examples, the tilt may include tilts in the left and/or right directions. The asymmetric axial tilt reduces axial rotation deformity from positioning.

In regard to tilt, as noted above, with additional to reference to FIGS. 4A-4E, the tilt assembly 400 drives axial tilt (e.g., vertical directions) relative to the longitudinal axis of the Jackson frame, with a worm gear connection. For example, the tilt assembly 400 is comprised of a housing 402, a geared swing plate 404, a worm 406, and support rollers 408 all of which may be of any suitable size or shape. The worm 406 may be driven either with a manual handle or a powered motor. This interfaces with the toothed/geared swing plate 404, driving the tilting motion.

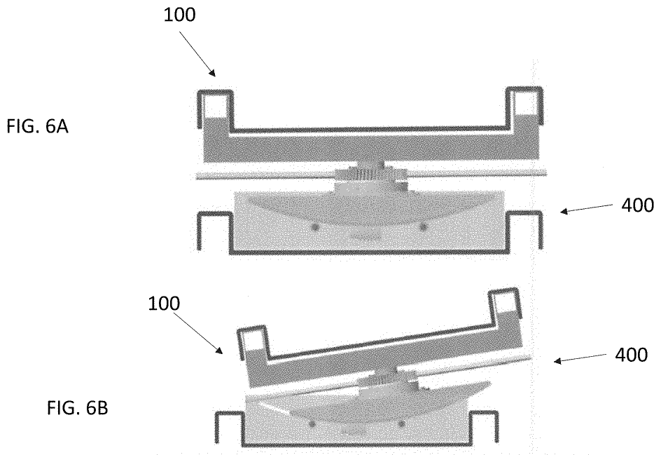

FIGS. 6A and 6B illustrate the break assembly 100 mounted to the tilt assembly 400, in accordance with examples of the present disclosure. The illustrated configurations allow for control of tilt (e.g., see FIGS. 4A-5C) and coronal break (e.g., see FIGS. 1A-3D).

For example, as shown on FIGS. 1A-2C, the first pad mount 102 and the second pad mount 104 may rotate independently relative to a longitudinal axis 106. This independent rotation allows for the coronal break to occur. In the neutral position, the mounts 102 and 104 may be aligned with one another along the axis 106 (e.g., both pads at 0 degrees of rotation). The worm gear connection, as illustrated in FIGS. 2A-2C allows rotation. For instance, each break assembly 100 may further include a base 202, a bearing 204, a worm gear 206, a worm 208, and a worm shaft 210. The bearing 204 connects the base 202 to the worm gear 206 allowing the worm gear 206 to rotate with respect to the base 202.

For example, with additional reference to FIGS. 4A-4E, the tilt assembly 400 is comprised of a housing 402, a geared swing plate 404, a worm 406, and support rollers 408 all of which may be of any suitable size or shape. The worm 406 may be driven either with a manual handle or a powered motor. This interfaces with the toothed/geared swing plate 404, driving the tilting motion. Rollers 408 support the load while allowing motion.

As best shown on FIG. 4C, the housing 402 includes a track 410 that mates with a guide feature 412 (e.g., protrusion/ridge) on the swing plate 404 which prevents the swing plate 404 from lifting up from the housing 402. As best shown on FIG. 4D, to facilitate the swinging motion, the geared swing plate 404 includes rails 414 on the outer side adjacent to the housing 402, and a worm gear 416 mounted along the center plane. A worm 418 is mounted in the center plane of the housing 402 to drive the geared swing plate 404.

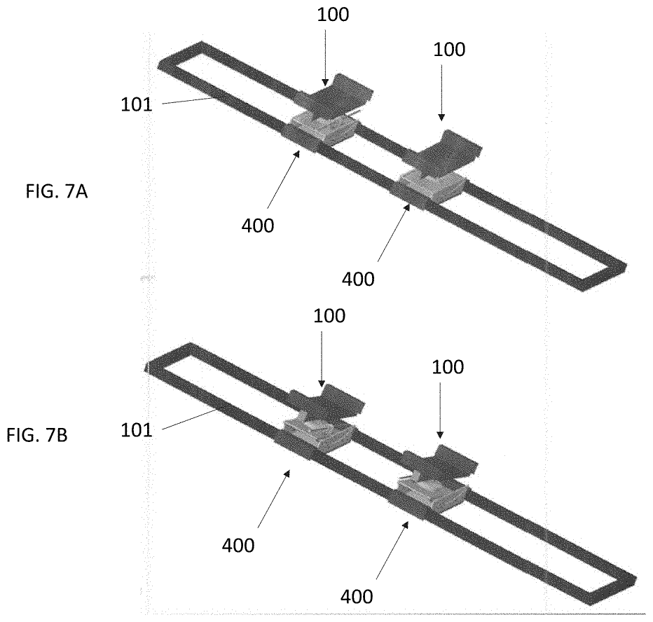

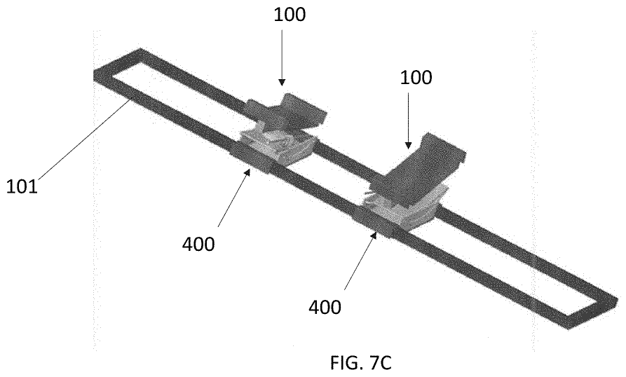

FIGS. 7A-7C illustrate the assemblies 100 and 400 mounted to the Jackson frame 101, in accordance with examples of the present disclosure. FIG. 7A illustrates the assemblies 100 and 400 in neutral positions. FIG. 7B illustrates the assemblies 100 and 400 in symmetric positions. FIG. 7C illustrates the assemblies 100 and 400 in asymmetric positions.

The break assembly 100 allows for rotation, while the tilt assembly 400 drives axial tilt (e.g., vertical directions) relative to the longitudinal axis of the Jackson frame, with a worm gear connection. For example, as shown on FIGS. 1A-2C, the first pad mount 102 and the second pad mount 104 may rotate independently relative to a longitudinal axis 106. This independent rotation allows for the coronal break to occur. In the neutral position, the mounts 102 and 104 may be aligned with one another along the axis 106 (e.g., both pads at 0 degrees of rotation).

The worm gear connection, as illustrated in FIGS. 2A-2C allows rotation to provide a coronal break. For instance, each break assembly 100 may further include a base 202, a bearing 204, a worm gear 206, a worm 208, and a worm shaft 210. The bearing 204 connects the base 202 to the worm gear 206 allowing the worm gear 206 to rotate with respect to the base 202.

The worm gear connection, as shown on FIGS. 4A-4E, allows tilt of a patient. For example, the tilt assembly 400 is comprised of a housing 402, a geared swing plate 404, a worm 406, and support rollers 408 all of which may be of any suitable size or shape. The worm 406 may be driven either with a manual handle or a powered motor. This interfaces with the toothed/geared swing plate 404, driving the tilting motion.

The described embodiments allow for control of tilt and coronal break of a patient during surgery with a frame such as a Jackson frame via worm gear connections. A worm gear connection may be used for coronal break and a second worm gear connection may be used for tilt.

Advantages may include coronal break to improve lateral access in the prone position. Also, axial tilt is improved for lateral access when the patient is in the prone position. The axial tilt allows ATP access in the prone position. The coronal break reduces scoliosis deformity from positioning. The asymmetric axial tilt reduces axial rotation deformity from positioning.

It is believed that the operation and construction of the present disclosure will be apparent from the foregoing description. While the apparatus and methods shown or described above have been characterized as being preferred, various changes and modifications may be made therein without departing from the spirit and scope of the disclosure as defined in the following claims.

What is claimed is:

1. A patient positioning system comprising:
a frame;
a set of break assemblies, each break assembly comprising a mount for receiving padding, wherein each break assembly is operable to independently rotate to provide a coronal break relative to a longitudinal axis of the frame; and
a set of tilt assemblies mounted to the frame, each tilt assembly operable to independently tilt relative to the longitudinal axis of the frame, wherein the break assemblies are mounted to the tilt assemblies.

2. The system of claim 1, wherein each break assembly further comprises the padding, the padding configured to receive a region of a patient.

3. The system of claim 1, wherein each break assembly comprises a worm operable to rotate a worm gear to provide the coronal break.

4. The system of claim 3, further comprising a shaft to rotate the worm.

5. The system of claim 4, further comprising a bearing adjacent to the worm gear.

6. The system of claim 5, further comprising a buffer plate adjacent to the bearing.

7. The system of claim 1, wherein each tilt assembly includes a swing plate operable to swing along a track.

8. The system of claim 7, wherein each swing plate includes a gear.

9. The system of claim 8, further comprising a second worm operable to contact the gear to move the swing plate along the track to adjust a tilt angle of the swing plate.

10. The system of claim 9, further comprising rollers to facilitate movement of the swing plate.

11. The system of claim 10, wherein the rollers and the worm are disposed below the swing plate.

12. A patient positioning system comprising:
a frame; and
a set of break assemblies, each break assembly comprising a mount for receiving padding, wherein each break assembly is operable to independently rotate to provide a coronal break relative to a longitudinal axis of the frame.

13. The system of claim 12, wherein each break assembly further comprises the padding, the padding configured to receive a region of a patient.

14. The system of claim 13, wherein each break assembly comprises a worm operable to rotate a worm gear to provide the coronal break between the mounts.

15. The system of claim 12, further comprising a shaft configured to rotate the worm.

16. The system of claim 15, further comprising a bearing adjacent to the worm gear.

17. A patient positioning system comprising:
a frame; and
a set of tilt assemblies mounted to the frame, each tilt assembly operable to independently tilt to adjust a padding mount relative to a longitudinal axis of the frame.

18. The system of claim 17, further comprising a set of break assemblies, each break assembly operable to independently rotate to provide a coronal break.

19. The system of claim 17, wherein each tilt assembly includes a swing plate operable to swing along a track.

20. The system of claim 19, wherein each swing plate includes a gear.
